(19) United States
(12) Patent Application Publication
(10) Pub. No.: US 2020/0261180 A1
(43) Pub. Date: Aug. 20, 2020

(54) 27-3SYSTEMS, METHODS, AND COMPUTER-READABLE MEDIA FOR PROVIDING STEREOSCOPIC VISUAL PERCEPTION NOTIFICATIONS AND/OR RECOMMENDATIONS DURING A ROBOTIC SURGICAL PROCEDURE

(71) Applicant: Covidien LP, Mansfield, MA (US)

(72) Inventor: Dwight Meglan, Westwood, MA (US)

(21) Appl. No.: 16/644,907

(22) PCT Filed: Sep. 5, 2018
(86) PCT No.: PCT/US2018/049457

§ 371 Date: Mar. 5, 2020

Related U.S. Application Data
(60) Provisional application No. 62/554,765, filed on Sep. 6, 2017.

(51) Int. Cl. A61B 90/00; A61B 34/00; A61B 1/00

(52) U.S. Cl. CPC A61B 90/361; A61B 34/25; A61B 2090/365; A61B 2090/371; A61B 1/00009

(57) ABSTRACT

Provided in accordance with embodiments of the present disclosure are systems, methods, and computer-readable media for providing stereoscopic visual perception notifications and/or recommendations during a robotic surgical procedure. An exemplary method includes receiving a right-eye view image captured by way of a right-eye lens of a patient image capture device disposed at a surgical site, receiving a left-eye view image captured by way of a left-eye lens of the patient image capture device, analyzing the right-eye view and left-eye view images, determining, based on a result of the analyzing, whether the right-eye view image or the left-eye view image includes a characteristic, generating a stereoscopic visual perception notification, when it is determined that the right-eye view image or the left-eye view image includes the characteristic, and displaying a stereoscopic image based on the right-eye view image and the left-eye view image, the stereoscopic image including the stereoscopic visual perception notification.

### BACKGROUND

Robotic surgical systems have been used in minimally invasive surgical procedures. During a robotic surgical procedure, a surgeon controls one or more robotic surgical arms with a user interface at a remote console. The user interface allows the surgeon to manipulate a surgical instrument coupled to the robotic arm and to control a camera to receive images of a surgical site within a patient.

The console may include a stereoscopic display, sometimes referred to as a three-dimensional (3D) display. Such displays facilitate depth perception in an image by presenting the image to the surgeon as a pair of distinct images separately provided to the left and right eyes, respectively. The stereoscopic display may receive images provided by a stereoscopic endoscope employing two image signal pathways, usually dedicated to each of left-eye view images and right-eye view images, which are matched to simulate a stereoscopic image. During a surgical procedure, various factors may cause the stereoscopic images received from the stereoscopic endoscope to have one or more characteristic(s) that hinder stereoscopic visual perception, for example owing to a mismatch between an image received by way of the left-eye signal path and an image received by way of the right-eye signal path. Additional examples of factors that may cause the stereoscopic images to have one or more characteristics that hinder stereoscopic visual perception are described in Toward an Objective Stereo-Video Quality Metric: Depth Perception of Textured Areas, by Mikhail Erofeev, Dmitriy Vatolin, Alexander Voronov, Alexey Fedorov (International Conference on 3D Imaging (IC3D), pp. 1-6), 2012; Automatic Left-Right Channel Swap Detection, by D. Akimov, A. Shestov, A. Voronov, D. Vatolin, (International Conference on 3D Imaging (IC3D), pp. 1-6), 2012; System for Automatic Detection of Distorted Scenes in Stereo Video, by A. Voronov, A. Borisov, D. Vatolin, (Proceedings of the Sixth International Workshop on Video Processing and Quality Metrics (VPQM)), 2012; Towards Automatic Stereo-video Quality Assessment and Detection of Color and Sharpness Mismatch, by A. Voronov, D. Vatolin, D. Sumin, V. Napadovsky, A. Borisov, (International Conference on 3D Imaging (IC3D), pp. 1-6), 2012; Methodology for stereoscopic motion picture quality assessment, by A. Voronov, D. Vatolin, D. Sumin, V. Napadovsky, and A. Borisov, (Proc. SPIE 8648, Stereoscopic Displays and Applications XXIV, vol. 8648, pp. 864810-1-864810-14), March 2013; and Automatic detection of artifacts in converted S3D video, by A. Bokov, D. Vatolin, A. Zachesov, A. Belous, and M. Erofeev, (Proc. SPIE 9011, Stereoscopic Displays and Applications XXV, vol. 9011, pp. 901112-1-901112-14), March 2014; the entire contents of each of which are incorporated herein by reference.

### SUMMARY

Provided in accordance with an embodiment of the present disclosure are methods for providing stereoscopic visual perception notifications and/or recommendations during a robotic surgical procedure. In an aspect of the present disclosure, an exemplary method includes receiving a right-eye view image captured by way of a right-eye lens of a patient image capture device disposed at a surgical site, receiving a left-eye view image captured by way of a left-eye lens of the patient image capture device disposed at the surgical site, analyzing the right-eye view and left-eye view images, determining, based on a result of the analyzing, whether the right-eye view image or the left-eye view image includes a characteristic, generating a stereoscopic visual perception notification, when it is determined that the right-eye view image or the left-eye view image includes the characteristic, and displaying a stereoscopic image based on the right-eye view image and the left-eye view image, the stereoscopic image including the stereoscopic visual perception notification.

In another aspect of the present disclosure, the method further includes identifying a cause of the characteristic included in the right-eye view image or the left-eye view image.

In a further aspect of the present disclosure, the identifying the cause of the characteristic included in the right-eye view image or the left-eye view image includes determining that the characteristic is associated with an image capture device factor.

In another aspect of the present disclosure, the identifying the cause of the characteristic included in the right-eye view image or the left-eye view image includes determining that the characteristic is associated with a system latency factor.

In yet another aspect of the present disclosure, the identifying the cause of the characteristic included in the right-eye view image or the left-eye view image includes determining that the characteristic is associated with a surgical site factor.

In still another aspect of the present disclosure, the identifying the cause of the characteristic included in the right-eye view image or the left-eye view image includes detecting at least one of a binocular disparity, a color imbalance, a sharpness imbalance, a focus mismatch, a depth discontinuity, or a scale mismatch.

In yet another aspect of the present disclosure, the method further includes providing a recommendation of how to correct the characteristic included in the right-eye view image or the left-eye view image.

In a further aspect of the present disclosure, the recommendation is based on the cause of the characteristic.

In another aspect of the present disclosure, at least one of the generating the stereoscopic visual perception notification or the providing the recommendation includes displaying a visual indication.

In a further aspect of the present disclosure, the displaying the visual indication includes displaying a message indicating the cause of the characteristic.

In another aspect of the present disclosure, at least one of the generating the stereoscopic visual perception notification or the providing the recommendation includes providing an audible signal.

In yet another aspect of the present disclosure, the generating the stereoscopic visual perception notification includes generating a tactile vibration.

In still another aspect of the present disclosure, the patient image capture device is a stereoscopic endoscope.

Provided in accordance with an embodiment of the present disclosure are systems for providing stereoscopic visual perception notifications during a robotic surgical procedure. In an aspect of the present disclosure, an exemplary system includes a patient image capture device including a right-eye lens and a left-eye lens, the patient image capture device being disposed at a surgical site and configured to capture a right-eye view image of the surgical site by way of the right-eye lens, and capture a left-eye view image of the surgical site by way of the left-eye lens. The system further includes a display device, at least one processor coupled to the patient image capture device, and a memory coupled to the at least one processor, the memory including instructions which, when executed by the at least one processor, cause the at least one processor to receive the captured right-eye view image and the captured left-eye view image, analyze the right-eye view and left-eye view images, determine, based on a result of the analyzing, whether the right-eye view image or the left-eye view image includes a characteristic, and generate a stereoscopic visual perception notification, when it is determined that the right-eye view image or the left-eye view image includes the characteristic, and cause the display device to display a stereoscopic image based on the right-eye view image and the left-eye view image, the stereoscopic image including the stereoscopic visual perception notification.

In a further aspect of the present disclosure, the instructions, when executed by the at least one processor, further cause the at least one processor to identify a cause of the characteristic included in the right-eye view image or the left-eye view image.

In yet a further aspect of the present disclosure, the instructions, when executed by the at least one processor, further cause the at least one processor to determine that the characteristic is associated with an image capture device factor.

In another aspect of the present disclosure, the instructions, when executed by the at least one processor, further cause the at least one processor to determine that the characteristic is associated with a system latency factor.

In yet another aspect of the present disclosure, the instructions, when executed by the at least one processor, further cause the at least one processor to determine that the characteristic is associated with a surgical site factor.

In still another aspect of the present disclosure, the instructions, when executed by the at least one processor, further cause the at least one processor to detect at least one of a binocular disparity, a color imbalance, a sharpness imbalance, a focus mismatch, a depth discontinuity, or a scale mismatch.

In yet another aspect of the present disclosure, the instructions, when executed by the at least one processor, further cause the at least one processor to provide a recommendation of how to correct the characteristic included in the right-eye view image or the left-eye view image.

In a further aspect of the present disclosure, the recommendation is based on the cause of the characteristic.

In another aspect of the present disclosure, the instructions, when executed by the at least one processor, cause the display device to display a visual indication of at least one of the stereoscopic visual perception notification or the recommendation.

In a further aspect of the present disclosure, the visual indication includes a message indicating the cause of the characteristic.

In another aspect of the present disclosure, at least one of the stereoscopic visual perception notification or the recommendation includes an audible signal.

In yet another aspect of the present disclosure, the stereoscopic visual perception notification includes a tactile vibration.

In still another aspect of the present disclosure, the patient image capture device is a stereoscopic endoscope.

Provided in accordance with an embodiment of the present disclosure are non-transitory computer-readable storage media storing instructions which, when executed by a processor, cause a computing device to receive a right-eye view image of a surgical site captured by way of a right-eye lens, receive a left-eye view image of the surgical site captured by way of a left-eye left, analyze the right-eye view and left-eye view images, determine, based on a result of the analyzing, whether the right-eye view image or the left-eye view image includes a characteristic, generate a stereoscopic visual perception notification, when it is determined that the right-eye view image or the left-eye view image includes the characteristic, and cause a display device to display a stereoscopic image based on the right-eye view image and the left-eye view image, the stereoscopic image including the stereoscopic visual perception notification.

In a further aspect of the present disclosure, the instructions, when executed by the processor, further cause the processor to identify a cause of the characteristic included in the right-eye view image or the left-eye view image.

In yet a further aspect of the present disclosure, the instructions, when executed by the processor, further cause the processor to determine that the characteristic is associated with an image capture device factor.

In another aspect of the present disclosure, the instructions, when executed by the processor, further cause the processor to determine that the characteristic is associated with a system latency factor.

In yet another aspect of the present disclosure, the instructions, when executed by the processor, further cause the processor to determine that the characteristic is associated with a surgical site factor.

In still another aspect of the present disclosure, the identifying the cause of the characteristic included in the right-eye view image or the left-eye view image includes detecting at least one of a binocular disparity, a color imbalance, a sharpness imbalance, a focus mismatch, a depth discontinuity, or a scale mismatch.

In yet another aspect of the present disclosure, the instructions, when executed by the processor, further cause the processor to provide a recommendation of how to correct the characteristic included in the right-eye view image or the left-eye view image.

In a further aspect of the present disclosure, the recommendation is based on the cause of the characteristic.

In another aspect of the present disclosure, the instructions, when executed by the processor, further cause the display device to display a visual indication of at least one of the stereoscopic visual perception notification or the recommendation.

In a further aspect of the present disclosure, the visual indication includes a message indicating the cause of the characteristic.

In another aspect of the present disclosure, at least one of the stereoscopic visual perception notification or the recommendation includes an audible signal.

Any of the above aspects and aspects of the present disclosure may be combined without departing from the scope of the present disclosure.

### BRIEF DESCRIPTION OF THE DRAWINGS

Objects and features of the presently disclosed systems, methods, and computer-readable media will become apparent to those of ordinary skill in the art when descriptions of various embodiments thereof are read with reference to the accompanying drawings, wherein:

FIG. 1 is a schematic diagram of an example robotic system including a user interface, in accordance with the present disclosure;

FIG. 2 is a simplified perspective view of a patient image capture device and a surgical instrument, in accordance with an embodiment of the present disclosure;

FIG. 3 illustrates a stereoscopic image of a surgical site as viewed by way of a patient image capture device, in accordance with the present disclosure;

FIGS. 4-7 illustrate example images of the surgical site of FIG. 3, as captured by way of a right-eye view or a left-eye view of the patient image capture device of FIG. 1 and/or FIG. 2, in accordance with the present disclosure; and

FIG. 8 is a flowchart illustrating an example method of providing stereoscopic visual perception notifications and/or recommendations during a robotic surgical procedure, in accordance with the present disclosure.

### DETAILED DESCRIPTION

The present disclosure generally relates to systems, methods, and computer-readable media for providing stereoscopic visual perception notifications and/or recommendations during a robotic surgical procedure. During a robotic surgical procedure, a patient image capture device is used to continuously capture images of a surgical site. A stereoscopic image is displayed to a clinician based on right-eye view images captured by way of a right-eye lens (or right-eye signal path) of a stereoscopic endoscope and a left-eye view image captured by way of a left-eye lens (or left-eye signal path) of the stereoscopic endoscope. Due to various factors, the received right-eye view image, the received left-eye view image, and/or the stereoscopic image may have one or more characteristic(s) that hinder stereoscopic visual perception. For example, stereoscopic visual perception may be hindered by environmental factors (such as anatomical material occluding a stereoscopic endoscope lens or an excessive proximity of a surgical instrument to a stereoscopic endoscope lens), design factors (such as increased latency of a computer system affecting display of images from one signal path more than the other while performing the robotic surgical procedure), and/or by other factors. By continuously or periodically monitoring and processing the received right-eye view images and the received left-eye view images during the robotic surgical procedure, the characteristic(s) that hinder stereoscopic visual perception can be detected, and notifications and/or recommendations may be dynamically provided, for example to enable the characteristic(s) to be minimized or avoided.

As used herein, the terms “clinician,” “surgeon,” and “observer,” generally refer to a user of a stereoscopic display device described herein. Additionally, although the terms “first eye” and “second eye” are used herein to refer to a left eye and a right eye, respectively, of a clinician, this use is provided by way of example and should not be construed as limiting. Throughout this description, the term “proximal” refers to the portion of the device or component thereof that is farthest away from the patient (and thus closest to the clinician and/or surgical robot) and the term “distal” refers to the portion of the device or component thereof that is closest to the patient (and thus furthest away from the clinician and/or surgical robot). Further, as referred herein, the term “signal path” (whether right-eye or left-eye) refers to an optical-electrical-optical signal path whereby images are captured optically, converted to an electrical/digital signal to be transmitted, and again converted back to an optical image when received by a computing or display device.

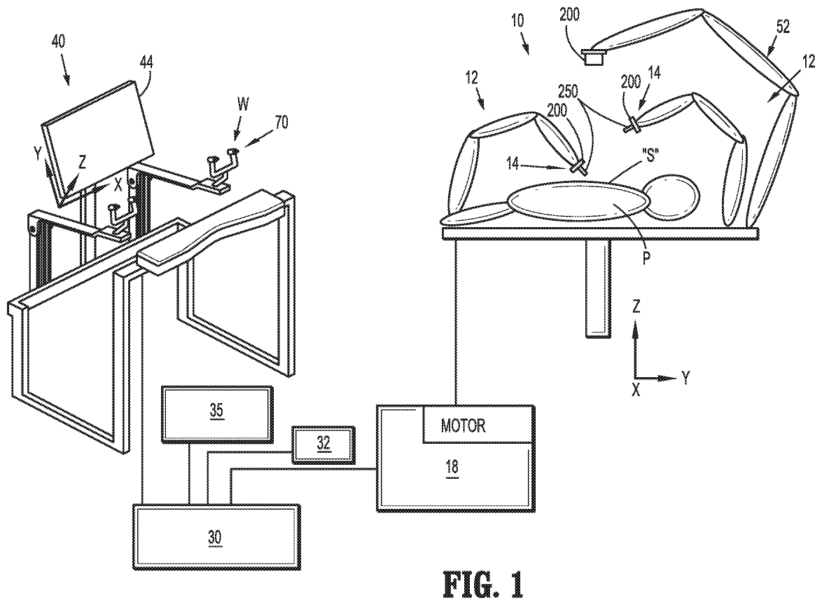

FIG. 1 shows an example robotic surgical system 1 in accordance with the present disclosure. The robotic surgical system 1 includes a surgical robot 10, a controller 30, a memory 35, and a user interface 40. Surgical robot 10 generally includes one or more robotic arms 12 and a base 18. Robotic arms 12 may be in the form of arms or linkages each having an end portion 14 that supports a surgical instrument 250. Surgical instrument 250 may be any type of instrument that may be used with robotic arm 12, such as an end effector, a grasper, a knife, scissors, and/or the like. One or more of robotic arms 12 may include a patient image capture device 200 for imaging a surgical site “S.”

Controller 30 includes, and/or is communicatively coupled to, one or more processors 32 (which, for convenience, may be referred to herein as “processor”) and memories 35 (which, for convenience, may be referred to herein as “memory”) and may be integrated with user interface 40 or provided as a separate, standalone device. As described in further detail below, processor 32 executes instructions stored in memory 35 to perform procedures of the various embodiments herein. As will be appreciated, the implementation of processor 32 and memory 35 is provided by way of example only and should not be construed as limiting. For instance, procedures of any of the embodiments of the present disclosure may be implemented by hardware components, firmware components, software components, and/or any combination thereof

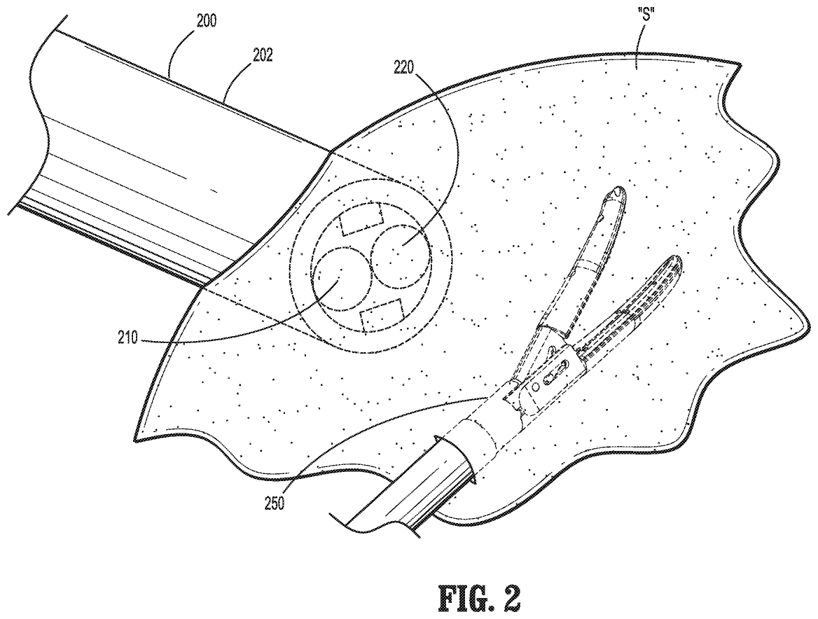

User interface 40 communicates with base 18 through controller 30 and includes a display device 44 which is configured to display stereoscopic images of the surgical site “S.” In embodiments, display device 44 may be an autostereoscopic display device, and/or a glasses-based stereoscopic display, such as, for example, anaglyph or polarization systems, or other passive stereoscopic display system. The images are captured by patient image capture device 200 and/or captured by imaging devices that are positioned about the surgical area (e.g., an imaging device positioned adjacent patient “P,” and/or positioned at a distal portion of an imaging arm 52). Patient image capture device 200 may capture visual images, infra-red images, ultrasound images, X-ray images, thermal images, and/or any other known real-time images of surgical site “S.” Patient image capture device 200 transmits captured images to controller 30. The captured images may then be processed and/or analyzed by processor 32, as described further below, and displayed by display device 44. The processing and/or analyzing of the captured images may occur prior to the captured images being displayed, thus potentially causing a slight delay in the captured images being displayed by display device 44. Alternatively, the processing and/or analyzing of the captured images may occur in real-time as the captured images are displayed by display device 44. In one embodiment, as further described below in the context of FIG. 2, the patient image capture device 200 is a stereoscopic endoscope capable of capturing images of at least a portion of the surgical site “S” by way of a right-eye lens 210 and a left-eye lens 220.

User interface 40 also includes input handles attached to gimbals 70 that allow a clinician to manipulate surgical robot 10 (e.g., move robotic arms 12, end portions 14 of robotic arms 12, and/or surgical instrument 250). Each gimbal 70 is in communication with controller 30 and processor 32 to transmit control signals thereto and to receive feedback signals therefrom. Additionally or alternatively, each gimbal 70 may include control interfaces or input devices (not shown) which allow the surgeon to manipulate (e.g., clamp, grasp, fire, open, close, rotate, thrust, slice, etc.) surgical instrument 250 supported at end portions 14 of robotic arms 12.

Each of gimbals 70 is moveable to move end portions 14 of robotic arms 12 within surgical site “S.” The stereoscopic images displayed on display device 44 are oriented such that movement of gimbals 70 moves end portions 14 of robotic arms 12 as viewed on display device 44. It will be appreciated that the orientation of the stereoscopic images on the display device may be mirrored or rotated relative to a view from above patient “P.” In addition, it will be appreciated that the size of the stereoscopic images on display device 44 may be scaled to be larger or smaller than the actual structures of surgical site “S” permitting the surgeon to have a better view of structures within surgical site “S.” As gimbal 70 is moved, surgical instruments 250 are moved within surgical site “S.” Movement of surgical instruments 250 may also include movement of end portions 14 of robotic arms 12 which support surgical instruments 250. In addition to gimbals 70, one or more additional input devices may be included as part of user interface 40, such as a handle including a clutch switch, a touchpad, joystick, keyboard, mouse, or other computer accessory, and/or a foot switch, pedal, trackball, or other actuatable device configured to translate physical movement from the clinician to signals sent to processor 32.

As noted briefly above, to provide the clinician with a view of surgical site “S” during a surgical procedure, patient image capture device 200 (such as a stereoscopic endoscope) may be disposed in surgical site “S” adjacent to surgical instrument 250 and configured to capture images of surgical site “S” to be displayed as stereoscopic images by way of display 44.

Turning now to FIG. 2, a simplified, perspective view of patient image capture device 200 and surgical instrument 250 is provided, in accordance with an embodiment of the present disclosure. Patient image capture device 200 captures right-eye view images of at least a portion of surgical site “S” via right-eye lens 210 and captures left-eye view images of at least a portion of the surgical site “S” via left-eye lens 220. Each set of a right-eye view image and a corresponding left-eye view image provides respective distinct viewpoint images that are transmitted to controller 30 to be processed and/or analyzed by processor 32, as described below, and to be displayed by display device 44. Patient image capture device 200 includes a body 202, which includes, at its distal portion, a lens assembly including right-eye lens 210 and left-eye lens 220. Right-eye lens 210 and left-eye lens 220 are positioned such that patient image capture device 200, using lenses 210, 220, is aligned with surgical site “S” and is able to continually capture images of surgical site “S”. For illustrative purposes, surgical instrument 250 is shown in some of the figures as a vessel sealing device.

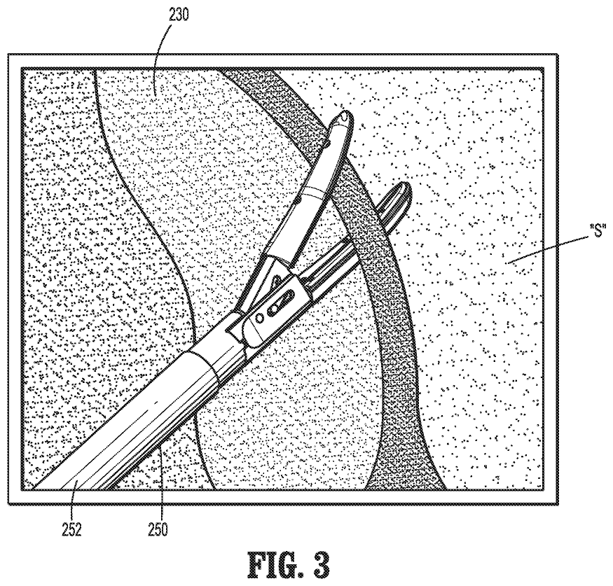

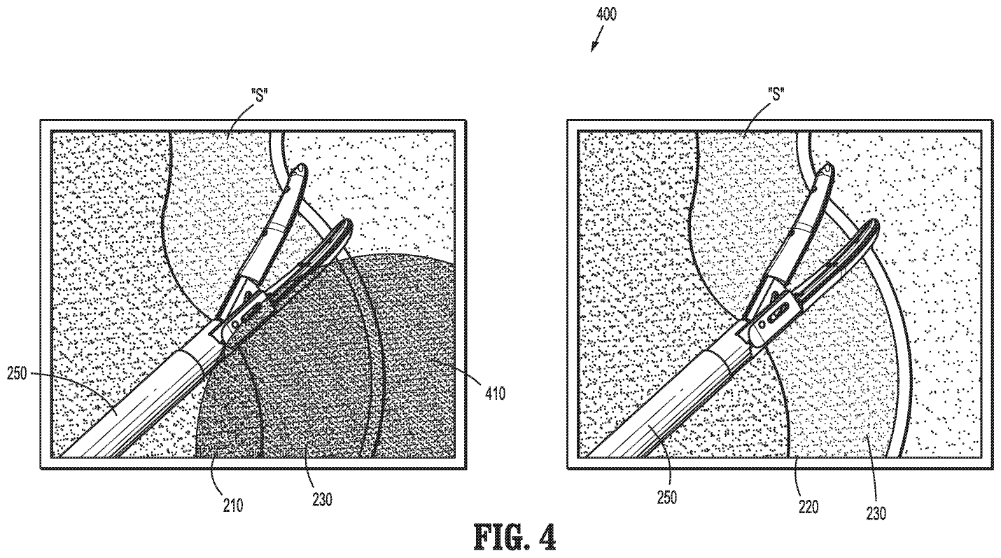

Referring now to FIG. 3, a stereoscopic image of surgical site “S” is illustrated. Surgical site “S” includes anatomical material 230, which may include tissue, bone, blood vessels, surgical procedure-related material and/or other biological material, and surgical instrument 250. Although shown as a single image, patient image capture device 200 receives two distinct images of surgical site “S,” captured via right-eye lens 210 and left-eye lens 220, respectively, as shown in FIGS. 4-7. It is further contemplated that processor 32 stores via memory 35 the right-eye view images and left-eye view images of surgical site “S” as the robotic surgical procedure is in progress.

As noted briefly above, images captured by patient image capture device 200 by way of right-eye lens 210 and left-eye lens 220 and/or the stereoscopic image displayed by display device 44 may have one or more characteristic(s) that hinder stereoscopic visual perception. For example, one or more characteristic(s) that hinder stereoscopic visual perception may include, without limitation, blurring of the stereoscopic images or other visual perception issues in the stereoscopic images, which may cause discomfort to a clinician viewing the stereoscopic images, or cause a clinician difficulty in resolving features of the stereoscopic images. One or more characteristic(s) that hinder stereoscopic visual perception may be associated with environmental factors (such as anatomical material occluding lenses 210, 220 or an excessive proximity of a surgical instrument 250 to lenses 210, 220), design factors (such as increased latency of a computer system and/or a delay caused by an electrical portion of a signal path affecting display of images from one signal path more so than the other while performing the robotic surgical procedure), and/or other factors. Some other factors may include a change in a physical aspect of patient image capture device 200 since it was last operating appropriately, which may include, without limitation, lens degradation, failure of one or both of lenses 210, 220, and/or incorrect zooming of one or both of lenses 210, 220. Examples of these factors are described in more detail below.

As shown in illustration 400 of FIG. 4, both images captured by way of right-eye lens 210 and left-eye lens 220 of surgical site “S” include surgical instrument 250 and anatomical material 230. Images received by way of right-eye lens 210 further include an image of occluding material 410 which is located on or in front of right-eye lens 210 and is partially obstructing the images captured by way of right-eye lens 210. Occluding material 410 may be anatomical material 230, surgical procedure-related material and/or other biological material, which completely or partially obstructs right-eye lens 210 and/or left-eye lens 220. Due to the presence of occluding material 410, the images captured via right-eye lens 210 may result in a characteristic that hinders stereoscopic visual perception, and therefore the stereoscopic visual perception of images formed from combining the right-eye view images and left-eye view images, received by way of the right-eye signal path and the left-eye signal path, respectively, may be compromised.

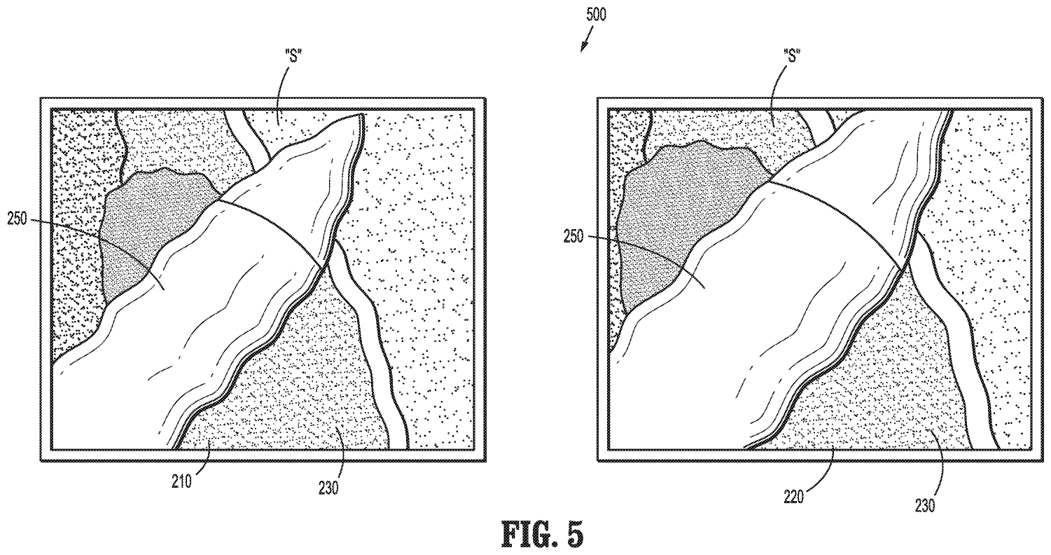

Referring now to FIG. 5, images captured by way of right-eye lens 210 and left-eye lens 220 are shown in illustration 500. As shown in FIG. 5, surgical instrument 250 appears to be enlarged and out of focus due to the proximity of patient image capture device 200 to surgical instrument 250, as illustrated by the blurring of images received by way of the right-eye signal path and the left-eye signal path. This is caused by patient image capture device 200 being too close to surgical instrument 250. Additionally, because of the location of patient image capture device 200, the focal point of the stereoscopic images is no longer located at surgical instrument 250, thereby distorting the binocular disparity in the stereoscopic images as perceived by the clinician from the images captured by way of lenses 210, 220. The disparate relative distance of surgical instrument 250 to right-eye lens 210 as compared to left-eye lens 220 may cause one or both of the clinician's eyes to rotate inward while viewing the images when attempting to focus on objects (e.g. surgical instrument 250) that are too close to one or both lenses 210, 220 of patient image capture device 200. This may cause discomfort and/or pain to the clinician, and, as in the example shown in FIG. 5, when an object such as surgical instrument 250 has disparate relative distances to right-eye lens 210 as compared to left-eye lens 220, the stereoscopic image may be distorted and the clinician may struggle to focus on both images simultaneously.

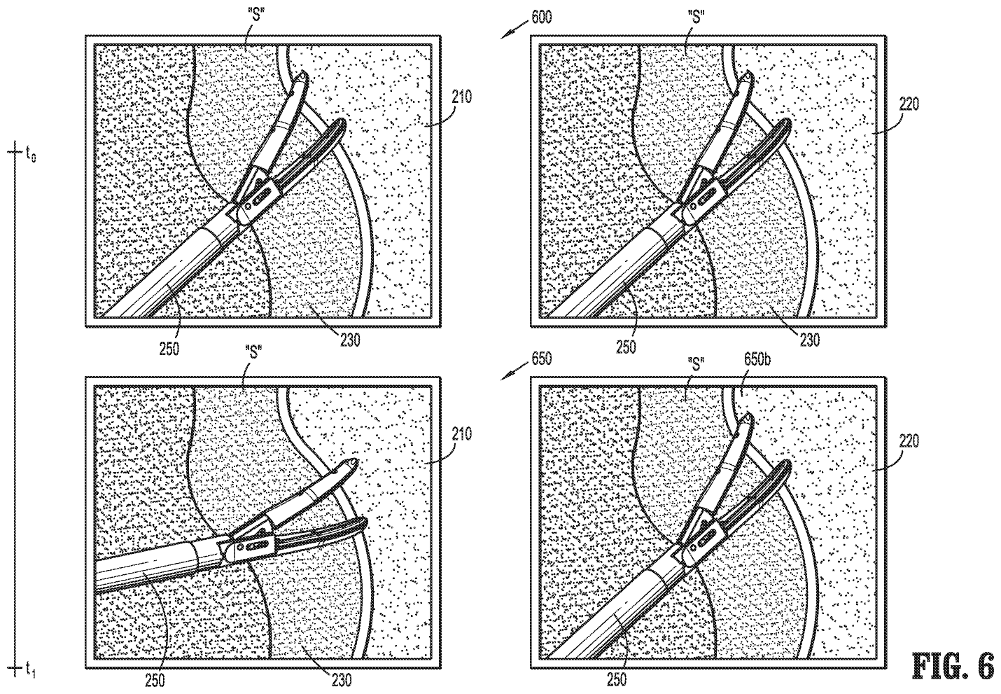

FIG. 6 illustrates a difference between images of surgical site “S” captured by way of right-eye lens 210 and left-eye lens 220 due to mismatched system latency issues affecting display of images from one signal path more so than the other. As is illustrated in FIG. 6, images captured by way of right-eye lens 210 and left-eye lens 220 are received by console 30 at two different times, for example, at initial time to, as shown in illustration 600 and at subsequent time ti occurring after initial time to, as shown in illustration 650. Images received by way of right-eye signal path and left-eye signal path at initial time to are matched. At subsequent time t₁, surgical instrument 250 as shown in the image received by way of the right-eye signal path appears to be located at a different location from surgical instrument 250 as shown in the image received by way of the right-eye signal path at initial time to, while surgical instrument 250 as shown in the image received by way of the left-eye signal path appears to be located at the same location as surgical instrument 250 as shown in the image received by way of the left-eye signal path at initial time to.

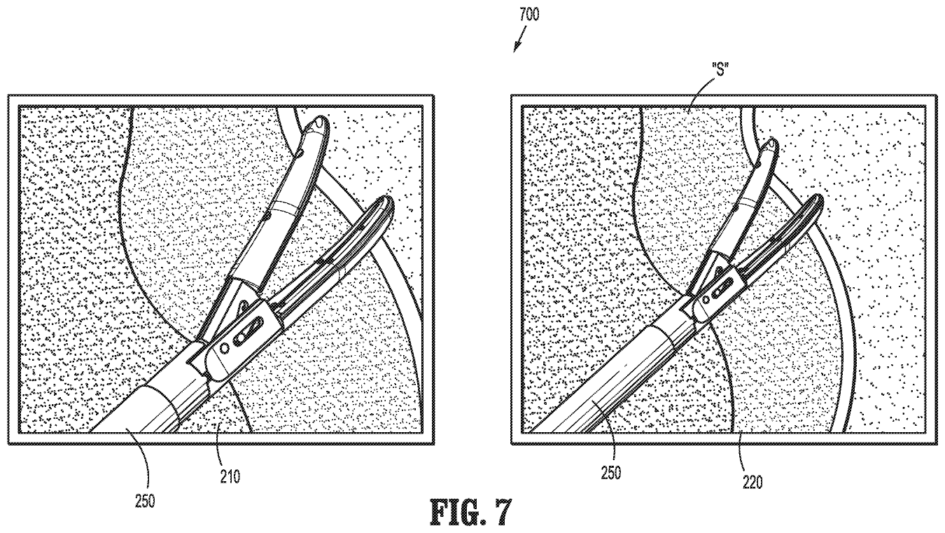

In another embodiment, an illustration 700 of surgical site “S” is shown in FIG. 7 where images captured by lenses 210, 220 each include surgical instrument 250 and anatomical material 230. As shown in FIG. 7, surgical instrument 250 in the images captured by right-eye lens 210 is much larger than surgical instrument 250 in the images captured by left-eye lens 220. The large difference between the images captured by lenses 210, 220 may be caused by a faulty right-eye lens 210 or the presence of anatomical material 230 or other material which has caused right-eye lens 210 to incorrectly be perceived as being at a different zoom level of surgical site “S.”

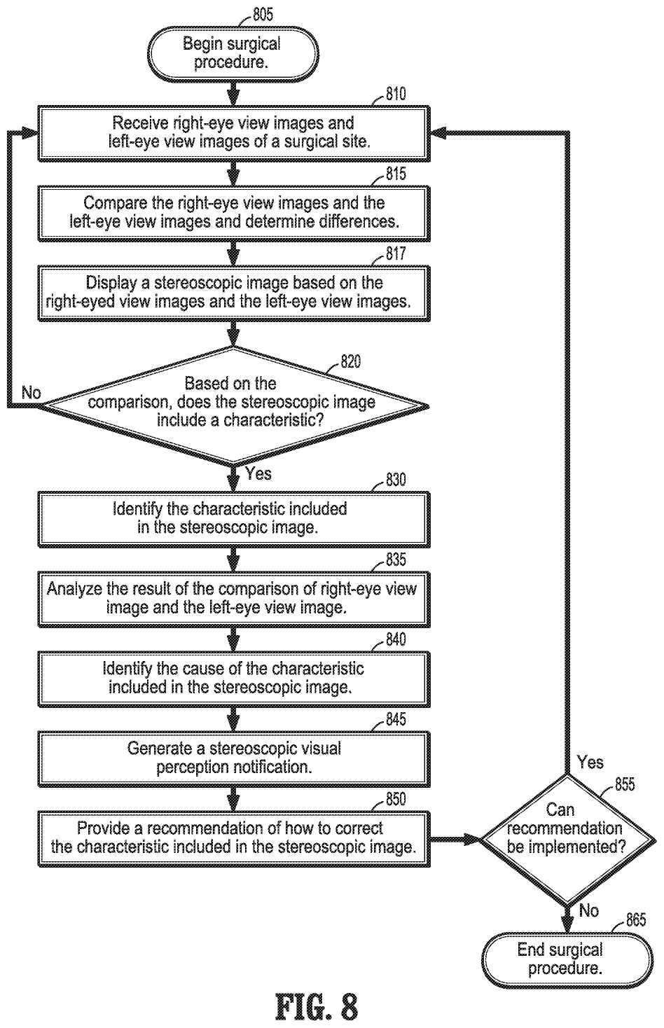

FIG. 8 is flowchart illustrating an illustrative method 800 of providing stereoscopic visual perception notifications and/or recommendations during a robotic surgical procedure, in accordance with embodiments of the present disclosure. Method 800 may be implemented, at least in part, by console 30, such as via processor 32 executing instructions stored in memory 35 (FIG. 1). Additionally, the particular sequence of steps shown in method 800 of FIG. 8 is provided by way of example and not limitation. Thus, the steps of method 800 may be executed in sequences other than the sequence shown in FIG. 8 without departing from the scope of the present disclosure. Furthermore, some steps shown in method 800 of FIG. 8 may be concurrently executed with respect to one another instead of sequentially executed with respect to one another, and some steps may be repeated and/or omitted without departing from the scope of the present disclosure.

Robotic surgical system 10 is set up to permit the clinician to begin the surgical procedure within surgical site “S,” at step 805. For example, the clinician moves the gimbals 70 to thereby position patient image capture device 200 and surgical instrument 250 in a manner to align the field of view of patient image capture device 200 with surgical site “S.”

Once suitably positioned, patient image capture device 200 captures (for example, continuously, intermittently, or periodically) a right-eye view image of surgical site “S” by way of right-eye lens 210 and a left-eye view image of surgical site “S” by way of left-eye lens 220, at step 810. By aligning the field of view of patient image capture device 200 with surgical site “S,” patient image capture device 200 is able to capture images of surgical site “S,” by way of lenses 210, 220. In addition to tissue and surrounding anatomical material 230 on which the procedure is being performed, the received images may include images of surgical instrument 250 as it is manipulated by the clinician.

At step 815, the images captured by way of lenses 210, 220 are transmitted to controller 30 via the right-eye signal path and the left-eye signal path, where the images are processed and/or analyzed. The processing and/or analysis of images captured by way of lenses 210, 220 and transmitted to console 30 via the right-eye signal path and left-eye signal path may be implemented by various image analysis algorithms and methods which cause controller 30, such as via processor 32, to determine differences between images received by way of the right-eye signal path and the left-eye signal path. For example, one such image analysis method may include sampling a plurality of pixels in corresponding areas of images received by way of the right-eye signal path and the left-eye signal path, and determining differences in color of the corresponding sampled pixels to identify reliable and unreliable images. For example, reliable images may be images where the color of the sampled pixels match or are within a predetermined color range of each other, while unreliable images may be images where the color of the sampled pixels are not within the predetermined color range of each other. In some embodiments, only portions or areas of images may be identified as unreliable—that is, a color mismatch may not be detected across an entire image, but rather just a portion of the image. For example, unreliable areas may include an excessive depth disparity in at least a portion of an image, and thus controller 30 may further calculate a disparity index for the reliable and unreliable areas and create a histogram of depth throughout the image to determine whether there is an excessive depth disparity between corresponding areas of the images received by way of the right-eye signal path and images received by way of the left-eye signal path. Controller 30 may also analyze the differences in color of the sampled pixels in the reliable images to determine whether a color mismatch is present in one or more areas of such reliable images. In embodiments, controller 30 may calculate a mean squared error threshold and compare the colors of the sampled pixels to the threshold to determine whether excessive color mismatch is present. Controller 30 may further determine whether a sharpness mismatch is present by performing a high frequency analysis of the pixel information across the images received by way of the right-eye signal path and the left-eye signal path to create a high frequency map of the images, and compare the high frequency maps of the images to determine whether a deviation above a predetermined threshold is present. In a further example, an image analysis method may include applying a modulation transfer function to portions of the images received by way of the right-eye signal path and the left-eye signal path, and determining that the stereoscopic visual perception of the stereoscopic images may be degraded. Those skilled in the art will recognize that various other image analysis algorithms may be used instead of or in addition to the algorithms described here without departing from the scope of the present disclosure.

Next, at step 817, controller 30 causes display device 44 to display a stereoscopic image based on (such as by combining) the right-eye view image and the left-eye view image received at step 5815. After step 817, method 800 proceeds to step 820 where controller 30 determines whether, based on the comparison and differences between images received by way of the right-eye signal path and the left-eye signal path, the stereoscopic image displayed by based on the right-eye view image and left-eye view image includes a characteristic. Characteristics included in the stereoscopic image include, but are not limited to, 3D blurring, color imbalance, incorrect focus, or other characteristics where the difference between images received by way of the right-eye signal path and the left-eye signal path is outside a range of the usual difference between images received by way of the right-eye signal path and the left-eye signal path necessary for the display of stereoscopic images by controller 30 and display device 44. If it is determined, at step 820, that, a characteristic is not included in the stereoscopic image (“No” at step 820), method 800 returns to step 810.

If it is determined, at step 820, that a characteristic is included in the stereoscopic image (“Yes” at step 820), method 800 proceeds to step 830. At step 830, the characteristic included in the stereoscopic image displayed by combining the left-eye view image and right-eye view image is identified. For example, the type of characteristic (e.g. 3D blurring, color imbalance, incorrect focus, etc.) may be identified. Next, at step 835, the result of the processing and/or analysis of the right-eye view image and the left-eye view image is analyzed and, at step 840, the cause of the characteristic is identified. A characteristic such as incorrect focus may be caused by a mismatch of images received by way of the right-eye signal path and the left-eye signal path and/or other problems related to images captured by lenses 210, 220 and include, but are not limited to, issues arising from vertical parallax, depth budget, depth continuity, binocular disparity, mismatch of scaling (as shown in FIG. 7), rotational mismatch (for example, where one or both of lenses 210, 220 are rotated), color mismatch, sharpness mismatch, channel mismatch (swapped views), stereo window violation perceptibility, temporal shift, crosstalk perceptibility, and the like. In an embodiment, memory 35 includes a database of stereoscopic image characteristics, the likely causes, and probable solutions/recommendations, and controller 30 determines the likely cause of the characteristic that hinders stereoscopic visual perception by using the processing and/or analysis of images received by way of the right-eye signal path and the left-eye signal path and by referring to the database. In addition, the characteristic included in the stereoscopic image may also be caused by surgical site issues, such as occlusion of one or both lenses 210, 220 by occluding material 410 (as shown in FIG. 4), system latency issues, such as lagging of images received by way of the right-eye signal path and/or the left-eye signal path 220 (as shown in FIG. 6), and imaging device issues, such as incorrect zooming of right-eye lens 210 and/or left-eye lens 220 (as shown in FIG. 7). For example, if, during the analysis of the images received by way of the right-eye signal path and the left-eye signal path, it is observed that a portion of the images received by way of the right-eye signal path and/or the left-eye signal path (and thus the stereoscopic images) appears darker than the remaining portions, controller 30, using this information and referring to the database, will determine that the cause of the darker portion of the images is likely caused by occluding material (as shown in FIG. 4). In an embodiment, controller 30, via memory 35, stores data detailing the identified characteristic that hinders stereoscopic visual perception and the cause.

Next, at step 845, a stereoscopic visual perception notification is generated identifying the characteristic included in the stereoscopic image and the cause. The notification may be displayed, such as via display device 44 of user interface 40, and/or may be provided audibly, such as via speakers (not shown), or tactilely, such as via gimbals 70. In embodiments, the pixels corresponding to the characteristic identified at step 817 are highlighted or otherwise indicated in the stereoscopic images displayed by display device 44. Following step 845, method 800 proceeds to step 850 where a recommendation is provided of how to correct the characteristic included in the stereoscopic images. For example, the recommendation provided may be in the form of a notification displayed via display device 44. In the embodiment shown in FIG. 4, the recommendation may include instructions to the clinician to remove and/or clean one or both of lenses 210, 220 in order to improve image quality. In an alternative embodiment where patient image capture device 200 is indicated as being compromised, the recommendation may include instructions to the clinician to replace the current patient image capture device 200 with an intact device. In still another embodiment, for example, as may be applied to the embodiment shown in FIG. 6, the recommendation may include instruction for the clinician to, via gimbal 70, move patient image capture device 200 away from surgical instrument 250 in order to better focus the images captured by lenses 210, 220.

Next, at step 855, a determination is made of whether the recommendation can be implemented during the robotic surgical procedure. The determination is based on the database of probable solutions/recommendations stored in memory 35. For example, if the recommendation requires the clinician to replace patient image capture device 200 due to defective or compromised lenses 210, 220, the robotic surgical procedure may be required to be stopped before the recommendation may be implemented. Alternatively, where the recommendation requires the clinician to move patient image capture device 200 away from objects in surgical site “S,” the recommendation may be implemented while the robotic surgical procedure remains in progress. If it is determined at step 855 that the recommendation can be implemented while the surgical procedure remains in progress (“Yes” at step 855), method 800 returns to step 810 where new images of surgical site “S” are received. If, at step 855, it is determined that the recommendation cannot be implemented while the surgical procedure remains in progress (“No” at step 855), method 800 proceeds to step 865 where the robotic surgical procedure ends. In another embodiment, where the recommendation of how to correct the characteristic cannot be implemented while the surgical procedure remains in progress, a notification may be provided via display device 44 to stop the robotic surgical procedure.

Referring back to the computer-readable media of FIG. 1, memory 35 includes any non-transitory computer-readable storage media for storing data and/or software that is executable by processor 32 and which controls the operation of controller 30. In an embodiment, memory 35 may include one or more solid-state storage devices such as flash memory chips. Alternatively or in addition to the one or more solid-state storage devices, memory 35 may include one or more mass storage devices connected to processor 32 through a mass storage controller (not shown) and a communications bus (not shown). Although the description of computer-readable media contained herein refers to a solid-state storage, it should be appreciated by those skilled in the art that computer-readable storage media can be any available media that can be accessed by processor 32. That is, computer readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium which can be used to store the desired information and which can be accessed by workstation 180.

Detailed embodiments of devices, systems incorporating such devices, and methods using the same have been described herein. However, these detailed embodiments are merely examples of the disclosure, which may be embodied in various forms. Therefore, specific structural and functional details disclosed herein are not to be interpreted as limiting, but merely as a basis for the claims and as a representative basis for allowing one skilled in the art to employ the present disclosure in virtually any appropriately detailed structure.

What is claimed is:

1. A method for providing stereoscopic visual perception notifications during a robotic surgical procedure, the method comprising:
receiving a right-eye view image captured by way of a right-eye lens of a patient image capture device disposed at a surgical site;
receiving a left-eye view image captured by way of a left-eye lens of the patient image capture device disposed at the surgical site;
analyzing the right-eye view and left-eye view images;
determining, based on a result of the analyzing, whether the right-eye view image or the left-eye view image includes a characteristic;
generating a stereoscopic visual perception notification, when it is determined that the right-eye view image or the left-eye view image includes the characteristic; and
displaying a stereoscopic image based on the right-eye view image and the left-eye view image, the stereoscopic image including the stereoscopic visual perception notification.

2. The method of claim 1, further comprising identifying a cause of the characteristic included in the right-eye view image or the left-eye view image.

3. The method of claim 2, wherein the identifying the cause of the characteristic included in the right-eye view image or the left-eye view image includes determining that the characteristic is associated with an image capture device factor.

4. The method of claim 2, wherein the identifying the cause of the characteristic included in the right-eye view image or the left-eye view image includes determining that the characteristic is associated with a system latency factor.

5. The method of claim 2, wherein the identifying the cause of the characteristic included in the right-eye view image or the left-eye view image includes determining that the characteristic is associated with a surgical site factor.

6. The method of claim 2, wherein the identifying the cause of the characteristic included in the right-eye view image or the left-eye view image includes detecting at least one of a binocular disparity, a color imbalance, a sharpness imbalance, a focus mismatch, a depth discontinuity, or a scale mismatch.

7. The method of claim 2, further comprising providing a recommendation of how to correct the characteristic included in the right-eye view image or the left-eye view image.

8. The method of claim 7, wherein the recommendation is based on the cause of the characteristic.

9. The method of claim 7, wherein at least one of the generating the stereoscopic visual perception notification or the providing the recommendation includes displaying a visual indication.

10. The method of claim 9, wherein the displaying the visual indication includes displaying a message indicating the cause of the characteristic.

11. The method of claim 7, wherein at least one of the generating the stereoscopic visual perception notification or the providing the recommendation includes providing an audible signal.

12. The method of claim 1, wherein the generating the stereoscopic visual perception notification includes generating a tactile vibration.

13. The method of claim 1, wherein the patient image capture device is a stereoscopic endoscope.

14-37. (cancelled)
